(19) United States
(12) Patent Application Publication
(10) Pub. No.: US 2023/0390018 A1
(43) Pub. Date: Dec. 7, 2023

(54) FORCE SENSITIVE MECHANISM FOR CONTACT DETECTION IN CATHETER SYSTEMS

(71) Applicant: Boston Scientific Scimed, Inc., Maple Grove, MN (US)

(72) Inventor: Nathan Paul Hagstrom, Minneapolis, MN (US)

(21) Appl. No.: 18/312,499

(22) Filed: May 4, 2023

Related U.S. Application Data
(60) Provisional application No. 63/348,078, filed on Jun. 2, 2022.

(51) Int. Cl. A61B 90/00; A61B 18/14

(52) U.S. Cl. CPC A61B 90/06; A61B 18/1492; A61B 2017/0011

(57) ABSTRACT

A catheter comprising a shaft and a distal end portion extending distally from the distal end of the shaft and defining a longitudinal axis. The distal end portion comprises a proximal segment, a distal segment located distally of the proximal segment, the distal segment being displaceable relative to the proximal segment, and a force sensing mechanism that includes a proximal housing fixed within the proximal segment, a piezoelectric sensor mounted to the proximal housing and having a first portion fixedly secured to the proximal housing, and a second portion that is not fixedly secured to the proximal housing, and a distal housing fixed within the distal segment and including a projection that contacts the second portion of the piezoelectric sensor and is configured to apply an axial force to the second portion of the piezoelectric sensor upon application of an external force to the distal segment.

### CROSS REFERENCE TO RELATED APPLICATIONS

This application claims the benefit of U.S. Provisional Patent Application No. 63/348,078, filed Jun. 2, 2022, which is incorporated herein by reference in its entirety.

### TECHNICAL FIELD

The present disclosure relates generally to various force sensing catheter features.

### BACKGROUND

In ablation therapy, it may be useful to assess the contact between the ablation element and the tissue targeted for ablation. In interventional cardiac electrophysiology (EP) procedures, for example, the contact can be used to assess the effectiveness of the ablation therapy being delivered. Other catheter-based therapies and diagnostics can be aided by knowing whether a part of the catheter contacts targeted tissue, and to what degree the part of the catheter presses on the targeted tissue. The tissue exerts a force back on the catheter, which can be measured to assess the contact and the degree to which the catheter presses on the targeted tissue.

There is a continuing need for improved catheter apparatuses capable of sensing force applied by the catheter tip to target tissue, and corresponding methods of use.

### SUMMARY

In Example 1, a catheter adapted to measure a contact force, the catheter comprising an elongate shaft having a proximal end and a distal end, and a distal end portion extending distally from the distal end of the shaft. The distal end portion defines a longitudinal axis extending therethrough and comprises a proximal segment, a distal segment located distally of the proximal segment, the distal segment being spaced from the proximal segment by a gap, and a force sensing mechanism. The force sensing mechanism comprises a proximal housing fixed within the proximal segment, a piezoelectric sensor mounted to the proximal housing and having a first portion fixedly secured to the proximal housing and a second portion that is not fixedly secured to the proximal housing, and a distal housing fixed within the distal segment and including a projection that contacts the second portion of the piezoelectric sensor and is configured to apply an axial force to the second portion of the piezoelectric sensor upon application of an external force to the distal segment. The piezoelectric sensor is configured to generate an output indicative of an amount of the axial force applied to the second portion of the piezoelectric sensor in response to the external force applied to the distal segment.

In Example 2, the catheter of Example 1, wherein the proximal housing includes an upper surface, a lower surface, and a cavity extending from the lower surface through the upper surface, and wherein the first portion of piezoelectric sensor is fixedly attached to the upper surface, and wherein the second portion of the piezoelectric sensor extends at least partially across the cavity.

In Example 3, the catheter of Example 2, wherein the piezoelectric sensor has an arcuate or rectangular profile when viewed from a direction parallel to the longitudinal axis.

In Example 4, the catheter of Example 2, wherein the piezoelectric sensor has an annular shape when viewed from a direction parallel to the longitudinal axis, and wherein the first portion is an outer circumferential portion of the piezoelectric sensor, and the second portion is located radially inward of the first portion of the piezoelectric sensor.

In Example 5, the catheter of Example 2, wherein the piezoelectric sensor is a generally circular disk, and wherein the second portion extends across the cavity.

In Example 6, the catheter of any of Examples 1-5, wherein the proximal housing comprises a compressible backing material disposed within a portion of the cavity and contacting the second portion of the piezoelectric sensor opposite the projection on the distal housing, wherein the backing material resists deformation of the second portion of the piezoelectric sensor when the external force is applied to the distal segment.

In Example 7, the catheter of any of Examples 1-6, wherein the force sensing mechanism comprises three piezoelectric sensors mounted to the proximal housing and circumferentially spaced about the longitudinal axis, each of the piezoelectric sensors having a first portion fixedly secured to the proximal housing, and a second portion that is deflectable relative to the first portion.

In Example 8, the catheter of Example 7, wherein the distal housing comprises three projections, each projection contacting the second portion of a respective one of the piezoelectric sensors and configured to apply an axial force to the second portion of the respective piezoelectric sensor upon application of the external force to the distal segment.

In Example 9, the catheter of Example 8, wherein each of the piezoelectric sensors is configured to generate an output indicative of an amount of the axial force applied to the second portion thereof in response to the external force applied to the distal segment.

In Example 10, the catheter of Example 9, wherein the proximal housing comprises three cavities extending from the lower surface through the upper surface, each of the cavities being aligned with a respective one of the piezoelectric sensors, wherein the second portion of each of the piezoelectric sensors extends at least partially across a respective one of the cavities.

In Example 11, the catheter of Example 10, wherein each piezoelectric sensor has an arcuate or rectangular profile when viewed from a direction parallel to the longitudinal axis.

In Example 12, the catheter of Example 10, wherein each piezoelectric sensor has an annular shape when viewed from a direction parallel to the longitudinal axis, and wherein the first portion is an outer circumferential portion of the piezoelectric sensor, and the second portion is located radially inward of the first portion of the piezoelectric sensor.

In Example 13, the catheter of Example 10, wherein each piezoelectric sensor is a generally circular disk, and wherein the second portion of each piezoelectric sensor extends across the respective cavity.

In Example 14, the catheter of any of Examples 8-13, wherein a compressible backing material is disposed within a portion of each cavity and contacts the second portion of the respective piezoelectric sensor opposite the respective projection on the distal housing, wherein the backing material resists deformation of the second portion of the respective piezoelectric sensor when the external force is applied to the distal segment.

In Example 15, the catheter of Example 1, further comprising a pre-load mechanism operatively coupled to the proximal housing and configured to allow a user to selectively pre-load the piezoelectric sensor.

In Example 16, a catheter adapted to measure a contact force, the catheter comprising an elongate shaft having a proximal end and a distal end, a distal end portion extending distally from the distal end of the shaft. The distal end portion defines a longitudinal axis extending therethrough and comprises a proximal segment, a distal segment located distally of the proximal segment, and a force sensing mechanism comprising a proximal housing fixed within the proximal segment, a piezoelectric sensor mounted to the proximal housing and having a first portion fixedly secured to the proximal housing and a second portion that is not fixedly secured to the proximal housing, and a distal housing fixed within the distal segment and including a projection that contacts the second portion of the piezoelectric sensor and is configured to apply an axial force to the second portion of the piezoelectric sensor upon application of an external force to the distal segment

In Example 17, the catheter of Example 16, wherein the proximal housing includes an upper surface, a lower surface, and a cavity extending from the lower surface through the upper surface, and wherein the first portion of piezoelectric sensor is fixedly attached to the upper surface, and wherein the second portion of the piezoelectric sensor extends at least partially across the cavity.

In Example 18, the catheter of Example 17, wherein the proximal housing comprises a compressible backing material disposed within a portion of the cavity and contacting the second portion of the piezoelectric sensor opposite the projection on the distal housing, wherein the backing material resists deformation of the second portion of the piezoelectric sensor when the external force is applied to the distal segment.

In Example 19, the catheter of Example 17, wherein the piezoelectric sensor has an arcuate or rectangular profile when viewed from a direction parallel to the longitudinal axis.

In Example 20, the catheter of Example 17, wherein piezoelectric sensor has an annular shape when viewed from a direction parallel to the longitudinal axis, and wherein the first portion is an outer circumferential portion of the piezoelectric sensor, and the second portion is located radially inward of the first portion of the piezoelectric sensor.

In Example 21, the catheter of Example 17, wherein the piezoelectric sensor is a generally circular disk, and wherein the second portion of each piezoelectric sensor extends across the respective cavity.

In Example 22, the catheter of Example 1, further comprising a pre-load mechanism operatively coupled to the proximal housing and configured to allow a user to selectively pre-load the piezoelectric sensor.

In Example 23, a catheter adapted to measure a contact force, the catheter comprising an elongate shaft having a proximal end and a distal end, a distal end portion extending distally from the distal end of the shaft. The distal end portion defines a longitudinal axis extending therethrough and comprises a proximal segment, a distal segment located distally of the proximal segment, and a force sensing mechanism comprising a proximal housing fixed within the proximal segment, the proximal housing having a lower surface and an upper surface, a plurality of piezoelectric sensors mounted to and circumferentially spaced from one another about the proximal housing, each piezoelectric sensor having a first portion fixedly secured to the proximal housing and a second portion that is not fixedly secured to the proximal housing, and a distal housing fixed within the distal segment and including a plurality of projections, each of the projections contacting the second portion of a respective one the piezoelectric sensors and being is configured to apply an axial force to the second portion of the respective piezoelectric sensor upon application of an external force to the distal segment, wherein each of the plurality of piezoelectric sensors is configured to generate an output indicative of an amount of the axial force applied to the second portion thereof in response to the external force applied to the distal segment.

In Example 24, the catheter of Example 23, wherein the proximal housing comprises a plurality of cavities extending from the lower surface through the upper surface, each of the cavities being aligned with a respective one of the piezoelectric sensors, wherein the second portion of each of the piezoelectric sensors extends at least partially across a respective one of the cavities.

In Example 25, The catheter of Example 24, wherein the proximal housing comprises a compressible backing material disposed within each of the cavities and contacting the second portion of the piezoelectric sensor positioned thereover opposite the respective projection on the distal housing, wherein the backing material resists deformation of the second portion of the piezoelectric sensor when the external force is applied to the distal segment.

In Example 26, the catheter of Example 24, wherein each piezoelectric sensor has an annular shape when viewed from a direction parallel to the longitudinal axis, and wherein the first portion is an outer radial portion of the piezoelectric sensor, and the second portion is an inner radial portion of the piezoelectric sensor.

In Example 27, the catheter of Example 24, wherein each piezoelectric sensor is a generally circular disk, and wherein the second portion of each piezoelectric sensor extends across the respective cavity.

In Example 28, the catheter of Example 24, wherein the force sensing mechanism comprises three piezoelectric sensors mounted to the proximal housing and circumferentially spaced about the longitudinal axis, each of the piezoelectric sensors having a first portion fixedly secured to the proximal housing, and a second portion that is deflectable relative to the first portion, and wherein the distal housing comprises three projections, each projection contacting the second portion of a respective one of the piezoelectric sensors and configured to apply an axial force to the second portion of the respective piezoelectric sensor upon application of the external force to the distal segment.

In Example 29, the catheter of Example 28, wherein the proximal housing comprises three cavities extending from the lower surface through the upper surface, each of the cavities being aligned with a respective one of the piezoelectric sensors, wherein the second portion of each of the piezoelectric sensors extends at least partially across a respective one of the cavities.

In Example 30, a force sensing mechanism for an ablation catheter, the force sensing mechanism adapted to measure a contact force and comprising a proximal housing having a lower surface and an upper surface, a plurality of piezoelectric sensors mounted to and circumferentially spaced from one another about the proximal housing, each piezoelectric sensor having a first portion fixedly secured to the proximal housing and a second portion that is not fixedly secured to the proximal housing, and a distal housing including a plurality of projections, each of the projections contacting the second portion of a respective one the piezoelectric sensors and being configured to apply an axial force to the second portion of the respective piezoelectric sensor upon application of an external force to the distal housing, wherein each of the plurality of piezoelectric sensors is configured to generate an output indicative of an amount of the axial force applied to the second portion thereof in response to the external force applied to the distal housing.

In Example 31, the force sensing mechanism of Example 30, wherein the proximal housing comprises a plurality of cavities extending from the lower surface through the upper surface, each of the cavities being aligned with a respective one of the piezoelectric sensors, wherein the second portion of each of the piezoelectric sensors extends at least partially across a respective one of the cavities.

In Example 32, the force sensing mechanism of Example 31, wherein the proximal housing comprises a compressible backing material disposed within each of the cavities and contacting the second portion of the piezoelectric sensor positioned thereover opposite the respective projection on the distal housing, wherein the backing material resists deformation of the second portion of the piezoelectric sensor when the external force is applied to the distal segment.

In Example 33, the force sensing mechanism of Example 30, wherein each piezoelectric sensor has a rectangular shape.

In Example 34, the force sensing mechanism of Example 30, wherein each piezoelectric sensor has an annular shape, wherein the first portion is an outer circumferential portion of the piezoelectric sensor, and the second portion is located radially inward of the first portion of the piezoelectric sensor.

In Example 35, the force sensing mechanism of Example 30, wherein each piezoelectric sensor is a generally circular disk, and wherein the second portion of each piezoelectric sensor extends across the respective cavity.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C show a system for measuring a force with a catheter in accordance with various embodiments of this disclosure.

FIG. 2 shows a block diagram of circuitry for controlling various functions described herein.

FIG. 3 shows a perspective view of a distal end of a catheter in accordance with various embodiments of this disclosure.

FIGS. 4A and 4B are perspective and elevation views, respectively, of a force sensing mechanism in the catheter of FIG. 3 in accordance with various embodiments of this disclosure.

FIG. 4C is a sectional elevation view, of the force sensing mechanism shown in FIG. 4A-4B in accordance with various embodiments of this disclosure.

FIGS. 5A, 5B and 5C are perspective, elevation and sectional elevation views, respectively, of an alternative force sensing mechanism for use in the catheter of FIG. 3 in accordance with various embodiments of this disclosure.

FIGS. 6A-6D are perspective, elevation and sectional elevation views of an alternative force sensing mechanism for use in the catheter of FIG. 3 in accordance with various embodiments of this disclosure.

FIGS. 7A and 7B are elevation and sectional views, respectively, of an alternative force sensing mechanism for use in the catheter of FIG. 3 in accordance with various embodiments of this disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Various cardiac abnormalities can be attributed to improper electrical activity of cardiac tissue. Such improper electrical activity can include, but is not limited to, generation of electrical signals, conduction of electrical signals, and/or mechanical contraction of the tissue in a manner that does not support efficient and/or effective cardiac function. For example, an area of cardiac tissue may become electrically active prematurely or otherwise out of synchrony during the cardiac cycle, thereby causing the cardiac cells of the area and/or adjacent areas to contract out of rhythm. The result is an abnormal cardiac contraction that is not timed for optimal cardiac output. In some cases, an area of cardiac tissue may provide a faulty electrical pathway (e.g., a short circuit) that causes an arrhythmia, such as atrial fibrillation or supraventricular tachycardia. In some cases, inactivate tissue (e.g., scar tissue) may be preferable to malfunctioning cardiac tissue.

Cardiac ablation is a procedure by which cardiac tissue is treated to inactivate the tissue. The tissue targeted for ablation may be associated with improper electrical activity, as described above. Cardiac ablation can lesion the tissue and prevent the tissue from improperly generating or conducting electrical signals. For example, a line, a circle, or other formation of lesioned cardiac tissue can block the propagation of errant electrical signals. In some cases, cardiac ablation is intended to cause the death of cardiac tissue and to have scar tissue reform over the lesion, where the scar tissue is not associated with the improper electrical activity. Lesioning therapies include electrical ablation, radiofrequency ablation, cryoablation, microwave ablation, laser ablation, and surgical ablation, among others. While cardiac ablation therapy is referenced herein as an exemplar, various embodiments of the present disclosure can be directed to ablation of other types of tissue and/or to non-ablation diagnostic and/or catheters that deliver other therapies.

Ideally, an ablation therapy can be delivered in a minimally invasive manner, such as with a catheter introduced into the heart through a vessel, rather than surgically opening the heart for direct access (e.g., as in a maze surgical procedure). For example, a single catheter can be used to perform an electrophysiology study of the inner surfaces of a heart to identify electrical activation patterns. From these patterns, a clinician can identify areas of inappropriate electrical activity and ablate cardiac tissue in a manner to kill or isolate the tissue associated with the inappropriate electrical activation. However, the lack of direct access in a catheter-based procedure may require that the clinician only interact with the cardiac tissue through a single catheter and keep track of all of the information that the catheter collects or is otherwise associated with the procedure. In particular, it can be challenging to determine the location of the therapy element (e.g., the proximity to tissue), the quality of a lesion, and whether the tissue is fully lesioned, under-lesioned (e.g., still capable of generating and/or conducting unwanted electrical signals), or over-lesioned (e.g., burning through or otherwise weakening the cardiac wall). The quality of the lesion can depend on the degree of contact between the ablation element and the targeted tissue. For example, an ablation element that is barely contacting tissue may not be adequately positioned to deliver effective ablation therapy. Conversely, an ablation element that is pressed too hard into tissue may deliver too much ablation energy or cause a perforation.

The present disclosure concerns, among other things, methods, devices, and systems for assessing a degree of contact between a part of a catheter (e.g., an ablation element) and tissue. Knowing the degree of contact, such as the magnitude and the direction of a force generated by contact between the catheter and the tissue, can be useful in determining the degree of lesioning of the targeted tissue. Information regarding the degree of lesioning of cardiac tissue can be used to determine whether the tissue should be further lesioned or whether the tissue was successfully ablated, among other things. Additionally or alternatively, an indicator of contact can be useful when navigating the catheter because a user may not feel a force being exerted on the catheter from tissue as the catheter is advanced within a patient, thereby causing vascular or cardiac tissue damage or perforation.

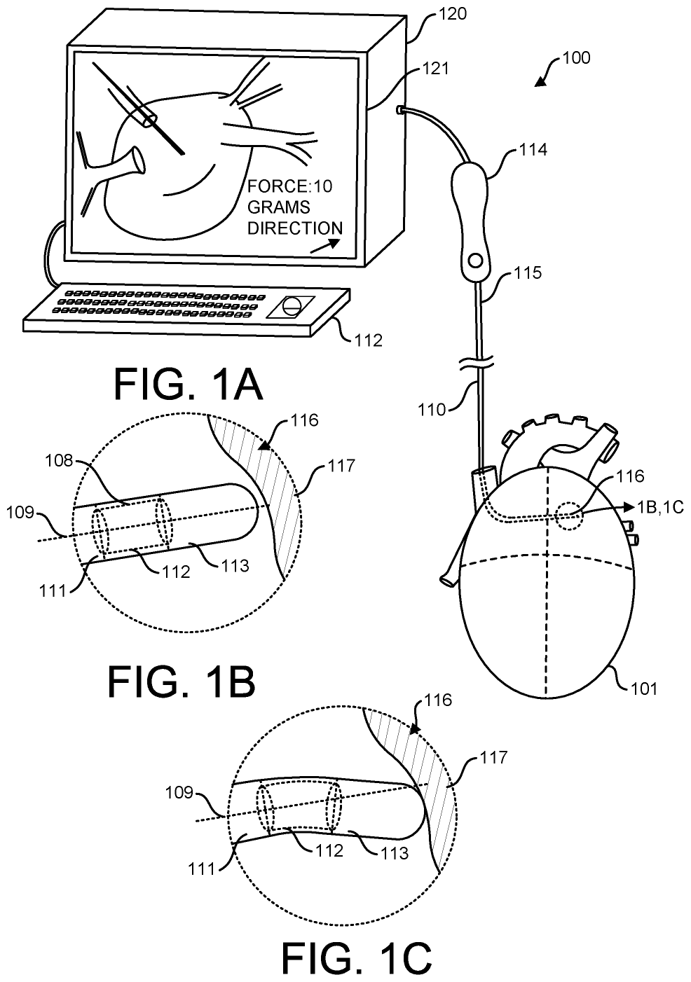

FIGS. 1A-1C illustrate an embodiment of a system 100 for sensing data from inside the body and/or delivering therapy. For example, the system 100 can be configured to map cardiac tissue and/or ablate the cardiac tissue, among other options. The system 100 includes a catheter 110 connected to a control unit 120 via handle 114. The catheter 110 can comprise an elongated tubular member having a proximal end 115 connected with the handle 114 and a distal end 116 configured to be introduced within a heart 101 or other area of the body. As shown in FIG. 1A, the distal end 116 of the catheter 110 is within the left atrium of the heart 101.

As shown in FIG. 1B, the distal end 116 of the catheter 110 includes a proximal segment 111, a force sensing mechanism 112, and a distal segment 113. The proximal segment 111 and the distal segment 113 can be coaxially aligned with each other in a base orientation as shown in FIG. 1B, and the force sensing mechanism 112 bridges the proximal and distal segments 111, 113. Specifically, in the illustrated embodiment, each of the proximal segment 111 and the distal segment 113 are coaxially aligned with a common longitudinal axis 109. In one embodiment, the longitudinal axis 109 can extend through the radial center of each of the proximal segment 111 and the distal segment 113, and can extend through the radial center of the distal end 116 as a whole. In some embodiments, the coaxial alignment of the proximal segment 111 with the distal segment 113 can correspond to the base orientation. As shown, the distal end 116, at least along the proximal segment 111 and the distal segment 113, extends straight. In some embodiments, this straight arrangement of the proximal segment 111 and the distal segment 113 can correspond to the base orientation.

The distal segment 113, or any other segment, can be in the form of an electrode configured for sensing electrical activity, such as electrical cardiac signals. In other embodiments, such an electrode can additionally or alternatively be used to deliver ablative energy to tissue.

In the various embodiments, force sensing mechanism 112 provides the catheter 110 with force sensing capabilities. For example, as shown in FIGS. 1B and 1C, the catheter 110 is configured to sense a force exerted upon the distal segment 113 due to engagement of the distal segment 113 with tissue 117 of heart 101. In various embodiments, the distal segment 113 can be relatively rigid, such that when the distal segment 113 engages tissue 117, forces applied to the tissue 117 by the tip of the distal segment 113 can be transferred to the proximal segment 111, and the magnitude and, in some embodiments, the direction of the applied forces can be sensed by the force sensing mechanism 112. As shown in FIGS. 1B and 1C, the applied force from the tissue causes relative displacement between the proximal segment 111 and the distal segment 113 (the skilled artisan will, based on the present disclosure, recognize that this relative displacement, which may be on the order of micrometers, is shown greatly exaggerated for illustration purposes in FIG. 1C). One or more sensors within the distal end 116 of the catheter 110 can sense the degree of bending or axial movement of the distal segment 113 relative to the proximal segment 111 to determine the magnitude and, in some embodiments, the direction of the applied force, as further discussed herein. When the distal segment 113 no longer engages the tissue 117, the proximal segment 111 and the distal segment 113 to the base orientation shown in FIG. 1B.

The control unit 120 of the system 100 includes a display 121 (e.g., a liquid crystal display or a cathode ray tube) for displaying information. The control unit 120 further includes a user input 122 which can include one or more buttons, toggles, a track ball, a mouse, touchpad, or the like for receiving user input. The user input 122 can additionally or alternatively be located on the handle 114. The control unit 120 can contain control circuitry for performing the functions referenced herein. Some or all of the control circuitry can alternatively be located within the handle 114.

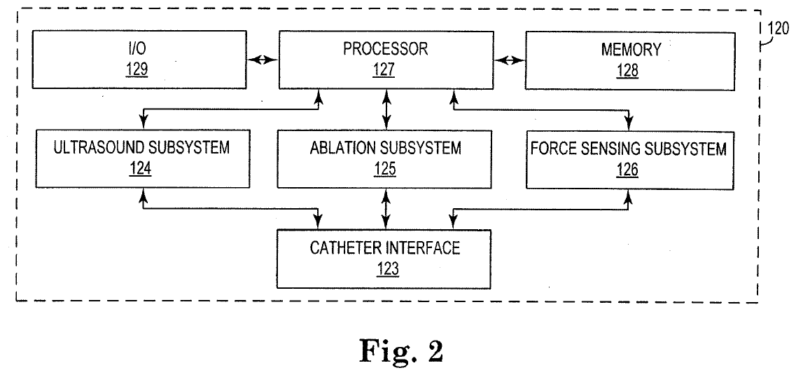

FIG. 2 illustrates a block diagram showing an example of control circuitry which can perform functions referenced herein. This or other control circuitry can be housed within control unit 120, which can comprise a single housing or multiple housings among which components are distributed. Control circuitry can additionally or alternatively be housed within the handle 114. The components of the control unit 120 can be powered by a power supply (not shown), as known in the art, which can supply electrical power to any of the components of the control unit 120 and the system 100. The power supply can plug into an electrical outlet and/or provide power from a battery, among other options.

The control unit 120 can include a catheter interface 123. The catheter interface 123 can include a plug which receives a cord from the handle 114. The catheter 110 can include multiple conductors (not illustrated but known in the art) to convey electrical signals between the distal end 116 and the proximal end 115 and further to the catheter interface 123. It is through the catheter interface 123 that the control unit 120 (and/or the handle 114 if control circuitry is included in the handle 114) can send electrical signals to any element within the catheter 110 and/or receive an electrical signal from any element within the catheter 110. The catheter interface 123 can conduct signals to any of the components of the control unit 120.

The control unit 120 can include hardware and software for use in imaging the tissue being mapped and/or treated. For example, in one embodiment, the control unit 120 can include an ultrasound subsystem 124 which includes components for operating the ultrasound functions of the system 100. While the illustrated example of control circuitry shown in FIG. 2 includes the ultrasound subsystem 124, it will be understood that not all embodiment may include ultrasound subsystem 124 or any circuitry for imaging tissue. The ultrasound subsystem 124 can include a signal generator configured to generate a signal for ultrasound transmission and signal processing components (e.g., a high pass filter) configured to filter and process reflected ultrasound signals as received by an ultrasound sensor in a sense mode and conducted to the ultrasound subsystem 124 through a conductor in the catheter 110. The ultrasound subsystem 124 can send signals to elements within the catheter 110 via the catheter interface 123 and/or receive signals from elements within the catheter 110 via the catheter interface 123.

It is emphasized, however, that the ultrasound subsystem 124, or other types of imaging subsystems, are strictly optional, and need not be included in the control unit 120.

The control unit 120 can include an ablation subsystem 125. The ablation subsystem 125 can include components for operating the ablation functions of the system 100. While the illustrated example of control circuitry shown in FIG. 2 includes the ablation subsystem, it will be understood that not all embodiment may include ablation subsystem 125 or any circuitry for generating an ablation therapy. The ablation subsystem 125 can include an ablation generator to provide different therapeutic outputs depending on the particular configuration. In one embodiment, the ablation generator is configured to generate a high frequency alternating current signal for delivering radiofrequency ablation energy to one or more electrodes. Alternatively, the ablation subsystem 125 and corresponding ablation generator may be configured to provide relatively high voltage pulses (monophasic or biphasic) to accomplish pulsed field ablation, thereby creating desired lesions in the target tissue via irreversible electroporation. The ablation subsystem 125 may support any other type of ablation therapy, such as microwave ablation. The ablation subsystem 125 can deliver signals or other type of ablation energy through the catheter interface 123 to the catheter 110.

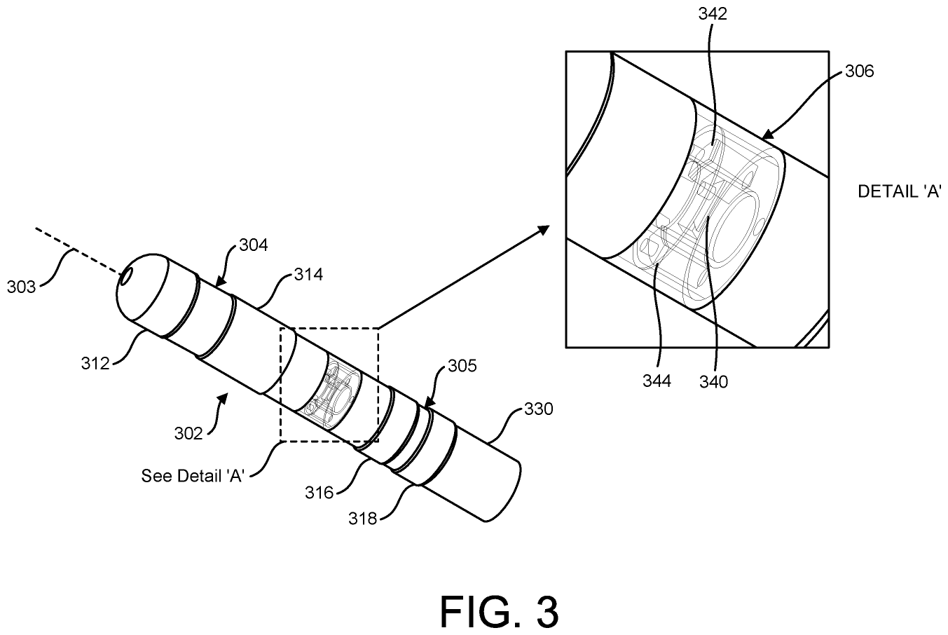

The control unit 120 can include a force sensing subsystem 126. The force sensing subsystem 126 can include components for measuring a force experienced by the catheter 110. Such components can include signal processors, analog-to-digital converters, operational amplifiers, comparators, and/or any other circuitry for conditioning and measuring one or more signals. The force sensing subsystem 126 can send electrical current to sensors, such as piezoelectric sensors (discussed below in reference to FIGS. 3-7B), within the catheter 110 via the catheter interface 123 and receive signals from sensors within the catheter 110 via the catheter interface 123.

Each of the ultrasound subsystem 124 (when present), the ablation subsystem 125, and the force sensing subsystem 126 can send signals to, and receive signals from, the processor 127. The processor 127 can be any type of processor for executing computer functions. For example, the processor 127 can execute program instructions stored within the memory 128 to carry out any function referenced herein, such as determine the magnitude and direction of a force experienced by the catheter 110.

The control unit 120 further includes an input/output subsystem 129 which can support user input and output functionality. For example, the input/output subsystem 129 may support the display 121 to display any information referenced herein, such as a graphic representation of tissue, the catheter 110, and a magnitude and direction of the force experienced by the catheter 110, amongst other options. Input/output subsystem 129 can log key and/or other input entries via the user input 122 and route the entries to other circuitry.

A single processor 127, or multiple processors, can perform the functions of one or more subsystems, and as such the subsystems may share control circuitry. Although different subsystems are presented herein, circuitry may be divided between a greater or lesser numbers of subsystems, which may be housed separately or together. In various embodiments, circuitry is not distributed between subsystems, but rather is provided as a unified computing system. Whether distributed or unified, the components can be electrically connected to coordinate and share resources to carry out functions.

FIG. 3 is an isometric illustration of a distal portion of a cardiac ablation catheter 300. In embodiments, the cardiac ablation catheter 300 corresponds to the ablation catheter 110 depicted in FIG. 1, and includes a distal assembly 302. As shown, the distal assembly 302 is disposed axially along a longitudinal axis 303 defined by the shaft (not shown in FIG. 2) of the ablation catheter 300. The distal assembly 302 includes a proximal segment 304, a distal segment 305, and a force sensing mechanism 306. The distal assembly 302 further includes a tip electrode 312 and a ring electrode 314, the tip electrode 312 being located at the distal end of the distal assembly 302, and the ring electrode 314 located proximal of and spaced apart from the tip electrode 312. In embodiments, the distal assembly 302 may include additional electrodes, e.g., electrodes 316, 318 disposed proximally of and longitudinally spaced from the electrodes 312 and 314. More or fewer electrodes may be employed in other embodiments within the scope of the present disclosure.

The particular operation of the various electrodes (or electrode pairs) can vary depending on the particular clinical use of the ablation catheter 300. In embodiments, the electrodes 312, 314, 316 and 318 may be configured to operate as ablation electrodes, sensing electrodes, or both. For example, any or all of the electrodes 312, 314, 316 and 318 can be configured to be operable for the delivery of ablative energy to target tissue. Additionally, or alternatively, any or all of the electrodes 312, 314, 316 and 318 can be operable as sensing electrodes configured to sense electrical signals (e.g., intrinsic cardiac activation signals and/or electric fields generated by injected currents for use in impedance-based location tracking, tissue proximity or contact sensing, and the like). In one embodiment, the electrodes 312, 314 may be configured to operate as ablation electrodes, e.g., for bi-polar delivery of ablation energy, and in particular, pulsed-field ablation energy for focal ablation of cardiac tissue. In embodiments, the electrodes 316, 318 may be operable as sensing electrodes, or alternatively, as ablation electrodes. In some instances, the electrodes 316, 318 are configured to measure local impedance, and may act as location sensors for sensing local electric fields in 5 degrees of freedom (e.g., 5 different motions—x, y, z, acceleration, and rotation). In embodiments, except as specifically described herein, the electrodes 312, 314, 316 and 318 may be configured in accordance with those described in co-pending and commonly-assigned U.S. Pat. App. 63/194,716, which is hereby incorporated by reference in its entirety.

It is emphasized, however, that the present disclosure is not limited to the particular electrode configurations and number of electrodes depicted in FIG. 3. Rather, the skilled artisan will appreciate that additional variations of electrode configurations, numbers of electrodes, and the like may be employed within the scope of the present disclosure.

In embodiments, the distal assembly 302 further includes an insulating material 330 encapsulating and forming an outer insulative surfaces of the proximal segment 304 and the distal segment 305. In embodiments, the insulating material 330 is formed by an overmolding process. Alternatively, the insulating material 330 can be formed using a reflow process in which one or more tubular segments of insulating material are disposed about the partially-assembled distal assembly 302 and then heated, as is known in the art. In embodiments, employing an overmolding process to provide the insulating material 330 can have certain advantages, e.g., to reduce or even eliminate the need for subsequent processing (such as the injection of medical adhesive to complete the assembly process and provide a fluid-tight connections between the various components). The insulating material may be commercially available Pebax® 55D and Pelathane® 55D. Both materials may be used in an overmolding process and bonded to an “epoxy bondable” wire insulation. Pellethane may adhere to the tip insulator using primer (e.g. Sivate™ E610) and plasma. Pebax may adhere to the tip insulator using adhesive (e.g. Thermedics 1-MP) without plasma.

As will be appreciated by those skilled in the art, although not shown in FIG. 3, in various embodiments the ablation catheter 300 may include additional components to enable its functionality. By way of example, in embodiments in which the ablation catheter 300 is of the deflectable or steerable type, the ablation catheter 300 can include structures, e.g., steering wires and associated anchor(s) to facilitate controlled deflection of the distal portion of the ablation catheter 300 by a user. Additionally, the ablation catheter 300 includes electrical conductors disposed within the catheter shaft to electrically couple the electrodes 312, 314, 316 and 318, the piezoelectric sensors 340, 342, 344, and other electrical components (e.g., magnetic navigation sensor(s) when present) to related functional components of the system 100. In general, constructional techniques and structures for implementing steerability or deflectability to ablation catheters and to electrically couple electrical components to the control unit of an ablation system are well known, and thus the skilled artisan will recognize that a wide range of such technologies can be employed in the ablation catheter 300.

In the various embodiments, the proximal segment 304 and the distal segment 305 are spaced from one another to allow for small deflections of the distal segment 305 relative to the proximal segment 304 when an external force is applied to the distal segment 305 by patient tissue, it being understood that such movement may be extremely small (e.g., on the order of micrometers). The force sensing mechanism 306 is operatively connected to both the proximal and distal segments 304, 305 and includes, in the illustrated embodiment, a plurality of piezoelectric sensors 340, 342, 344 circumferentially spaced from one another about the longitudinal axis 303. The sensors 340, 342, 344 are configured to generate a variable output based on the force applied by the distal segment 305 to target tissue, the output being calibrated to be indicative of the magnitude of such force.

In the embodiment of FIG. 3, the three piezoelectric sensors 340, 342, 344 are at evenly spaced azimuth angles about the longitudinal axis 303 (circumferentially arrayed evenly about the longitudinal axis 303) and at the same radial distance from the longitudinal axis 303. If the force exerted on the distal segment 305 of the catheter 300 is coaxial with the longitudinal axis 303, then the output from each of the piezoelectric sensors 340, 342, 344 will be substantially equal. Based on these equal changes, control circuitry can calculate a magnitude of the force exerted on the distal segment 306. The control circuitry can also determine that the force is coaxial with the longitudinal axis 303 because the output is the same for each of the three piezoelectric sensors 340, 342, 344.

If the force is not coaxial with the longitudinal axis 303, then the output for each of the piezoelectric sensors 340, 342, 344 will not be equal. Based on this, the magnitude and the direction (e.g., unit vector) of the force can be determined by the control circuitry.

Once assembled, the catheter 300 may undergo a calibration step, either at a factory or just before use by a physician. In such a step, a plurality of forces of known magnitude and direction can be placed, in sequence, on the distal segment 306, and the piezoelectric sensors 340, 342, 344 output can be calibrated based on the known magnitude and direction of the applied force.

The magnitude can be represented in grams or another measure of force. The magnitude can be presented as a running line graph, bar graph or graphic symbol varying with color or intensity that moves over time to show new and recent force values. The direction can be represented as a unit vector in a three dimensional reference frame (e.g., relative to an X, Y, and Z axes coordinate system). In some embodiments, a three dimensional mapping function can be used to track the three dimensional position of the distal end of the catheter 300 in the three dimensional reference frame. Magnetic fields can be created outside of the patient and sensed by a sensor (not shown) that is sensitive to magnetic fields within distal end of the catheter 300 to determine the three dimensional position and special orientation of the distal end of the catheter 300 in the three dimensional reference frame. The direction can be represented relative to the distal end of the catheter 300. For example, a line projecting to, or from, the distal segment 306 can represent the direction of the force relative to the distal segment 306. Similarly, a graphic symbol with varying color and/or intensity and/or shape could be utilized to represent the magnitude and/or the direction of the force. Such representations can be made on a display as discussed herein.

The magnitude and direction of the force can be used for navigation by providing an indicator when the catheter encounters tissue and/or for assessing the lesioning of tissue by determining the degree of contact between the lesioning element and the tissue, among other options. In some embodiments, a force under 10 grams is suboptimal for lesioning tissue (e.g., by being too small) while a force over 40 grams is likewise suboptimal for lesioning tissue (e.g., by being too large). Therefore, a window between 10 and 40 grams may be ideal for lesioning tissue and the output of the force during lesioning may provide feedback to the user to allow the user to stay within this window. Of course, other force ranges that are ideal for lesioning may be used.

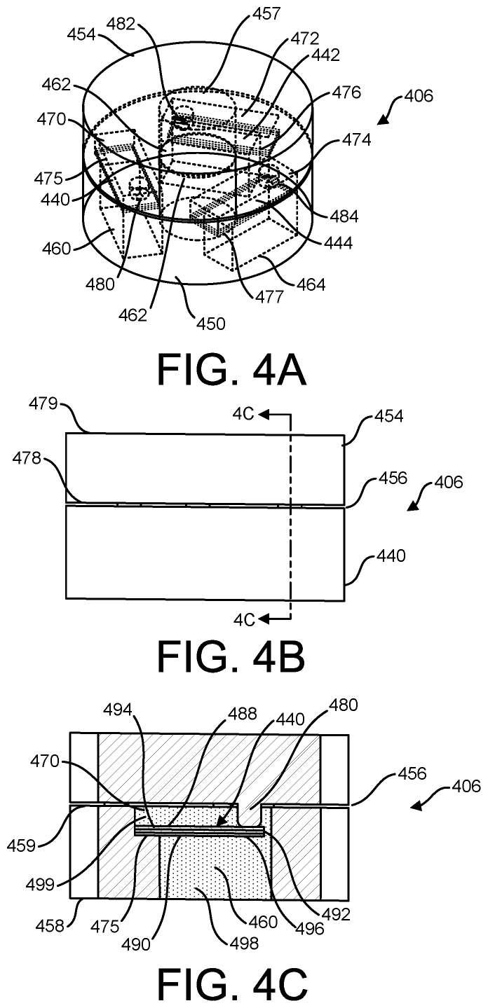

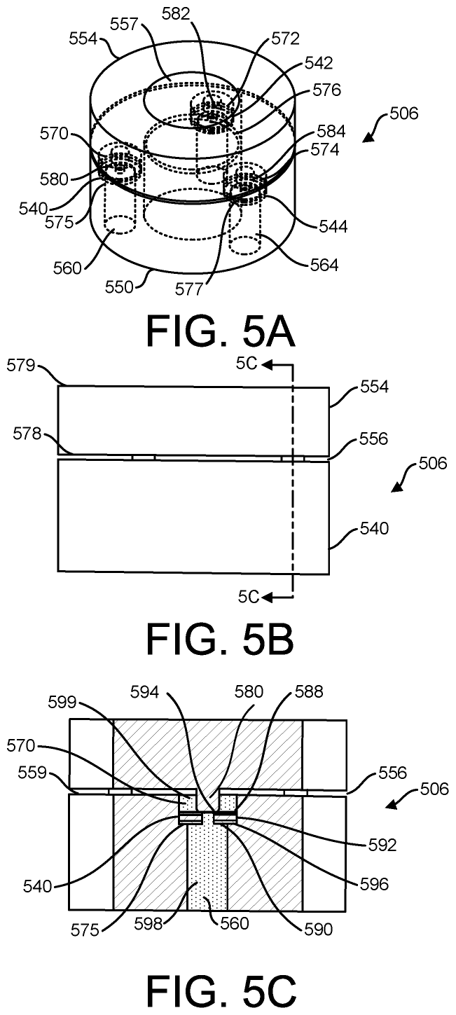

FIGS. 4A, 4B and 4C are perspective, elevation and cross-sectional views, respectively, of a force sensing mechanism 406 for use in the catheter 110, according to embodiments of the present disclosure. The force sensing mechanism 406 corresponds functionally to the force sensing mechanism 306 described above. In the illustrated embodiment, the force sensing mechanism 406 includes a plurality (in this case, three) piezoelectric sensors 440, 442, 444, a rigid proximal housing 450 and a rigid distal housing 454. As shown, the piezoelectric sensors 440, 442, 444 are disposed in and attached to the proximal housing 450. In the illustrated embodiment, the piezoelectric sensors 440, 442, 444 have a generally rectangular shape when viewed in a direction parallel to the axis of the catheter, although other embodiments utilize piezoelectric sensors having different shapes or form factors.

In the various embodiments, the proximal housing 450 is securely attached to the proximal segment (111 in FIG. 1, 304 in FIG. 3), and the distal housing 454 is securely attached to the distal segment (113 in FIG. 1, 306 in FIG. 3) of the catheters 110, 300 described above. Additionally, the proximal and distal housings 450, 454 are separated, in the absence of an external force on the distal segment of the respective catheter, by a gap 456, which defines the maximum axial movement of the distal segment relative to the proximal segment under the action of an external force. Also, in the illustrated embodiment, a lumen 457 extends longitudinally through both the proximal housing 450 and the distal housing 454 to accommodate passage of catheter components, e.g., conductor wires, irrigation tubing, and the like to the distal segment of the catheter.

FIG. 4C is a cross-sectional elevation view bisecting the force sensing mechanism 406 through the piezoelectric sensor 440. The particular structural details illustrated in FIG. 4C are representative of the arrangement of the piezoelectric sensors 442, 444. As shown in FIG. 4C, the proximal housing has a proximal face 458, an opposite distal face 459, a longitudinal cavity 460, and a distal recess 470 formed in the distal face 459, the recess 470 defining a shoulder 475. As further shown, the distal housing 454 has a proximal face 478, an opposite distal face 479, and includes an axial projection 480 extending proximally from the proximal face 478.

As shown in FIG. 4C, the piezoelectric sensor 440 includes an upper layer 488, a lower layer 490, and a piezoelectric layer 492 disposed therebetween. The piezoelectric sensor 440 further defines a fixed portion 494 and a free portion 496. As further shown, an optional backing element 498 is disposed within the longitudinal cavity 460 adjacent to the lower layer 490. Although for ease of illustration only the piezoelectric sensor 440 is shown in detail, it is emphasized that each of the piezoelectric sensors 442 and 444 have the same structure as the piezoelectric sensor 440. Additionally, the overall construction of the force sensing mechanism 406 with respect to the location of the piezoelectric sensors 442, 444 is identical to that shown in FIG. 4C, i.e., a cross-sectional elevation view bisecting the piezoelectric sensors 442, 444 will appear identical to the structures depicted in FIG. 4C, including the presence of a recess, longitudinal cavity, and projection and the corresponding relationship to the respective piezoelectric sensor.

The upper and lower layers 488, 490, or at least portions thereof, are electrically conductive and function as electrode layers during the operation of the piezoelectric sensor 440. Additionally, the piezoelectric layer 492 includes a piezoelectric material, for example, a piezoelectric ceramic such as lead zirconate titanate (PZT) or a piezoelectric polymer such as polyvinylidene fluoride (PVDF). As is recognized in the electro-mechanical arts, piezoelectric sensors generate measurable electrical characteristics when subjected to mechanical stress.

It is emphasized that the particular, e.g., 3-layer construction of the piezoelectric sensor 440 depicted herein is exemplary only and in now way intended to limit the scope of potential piezoelectric designs suitable for use in the force sensing mechanism 406. For example, in embodiments, the piezoelectric element itself can be a multi-layer or multi-piece construction. Additionally, the upper and lower layers 488, 490 may themselves be formed of multi-layer or multi-component construction. In embodiments, all or part of the piezoelectric sensors utilized herein may be coated with our encased in a protective and/or insulative outer coating, e.g., an epoxy.

With particular reference to FIG. 4C, the piezoelectric sensor 440 is positioned within the distal recess 470 of the proximal housing 450, with the fixed portion 494 positioned on the shoulder 475, and the free portion 496 extending over the longitudinal cavity 460. As further shown, the backing element 498 abuts the lower layer 490 along the free portion 496 of the piezoelectric sensor 440. Additionally, the axial projection 480 of the distal housing 454 is positioned over and bears upon the upper layer 488 in the free portion 496 of the piezoelectric sensor 440. The piezoelectric sensors 442, 444 each have the same positional relationship and with, respectively, a distal recess, a shoulder and a longitudinal cavity in the proximal housing 450, and an axial projection in the distal housing 454 as shown in FIG. 4C with respect to the piezoelectric sensor 440. Additionally, in embodiments, a backing element is disposed in the longitudinal cavity adjacent to the piezoelectric sensor 442, 444 in the same manner shown in FIG. 4C.

In embodiments, the fixed portion 494 of the piezoelectric sensor 440 is rigidly attached to the shoulder 475 of the proximal housing 450, and the free portion 496 of the piezoelectric sensor 440 is effectively cantilevered over the longitudinal cavity 460. Because the rigid axial projection 480 of the distal housing 454 is in direct contact with the upper layer 488 of the piezoelectric sensor 440, forces applied to the distal segment 305 (FIG. 3) of the catheter will be transmitted to the free portion 496 of the piezoelectric sensor 440. Additionally, the cantilevered arrangement of the free portion 496 will maximize the piezoelectric effect exhibited by the piezoelectric sensor 440 in response to the transmitted force (i.e., the stress induced in the piezoelectric layer 492, and the sensor output resulting from the corresponding piezoelectric effect, will be substantially greater than in an arrangement where the piezoelectric sensor is rigidly fixed along its entire length).

In embodiments, the backing element 498, when present, is made of a compressible material (e.g., an elastomer) and functions to provide some support to the free portion 496 of the piezoelectric sensor 440 to resist deformation thereof when the external force is applied, while at the same time does not rigidly resist all such deformation. In embodiments, the mechanical properties of the backing element 498 can be tailored to fine tune the piezoelectric effect exhibited by the piezoelectric sensor 440.

As will be appreciated by the skilled artisan, because the piezoelectric sensor 440 is an electro-mechanical devices, electrical leads (not shown) are attached to the electrode structures on the upper and lower layers 488, 490. In embodiments, the electrical leads may be routed through the longitudinal cavity 460, may be routed to the lumen 457, or through some other access features within the proximal housing 450. As further shown in FIG. 4C, in embodiments, a potting material 499 may be disposed within open spaces within the proximal housing 450, which can operate to hermetically seal the internal components of the catheter and the force sensing mechanism 406 and also to enhance the structural attachment between the proximal and distal segments of the catheter. In embodiments, the potting material 499 and the backing element 498 may be formed of the same compressible material, and may be formed in a single manufacturing step, although this is not strictly required. In some embodiments, the potting material 499 (or some other compressible material) may be disposed in the gap 456. In some embodiments, the backing element 498 and/or the potting material 499 may be omitted altogether.

It is emphasized again that the arrangement shown in FIG. 4C with respect to the piezoelectric sensor 440 is representative of the piezoelectric sensors 442 and 444.

As is generally recognized in the electro-mechanical arts, the piezoelectric sensors 440, 442, 444 will exhibit electrical properties that will vary as a function of stress developed within their respective piezoelectric layers. In embodiments, an alternating current electrical signal generated by the control unit 120 (FIG. 1A) may be delivered to the electrodes in the upper and lower layer of each piezoelectric sensor 440, 442, 444 and the changes in the response the electrical response exhibited by the piezoelectric layer in each sensor resulting from varying the stress therein caused by forces transferred thereto through the distal segment of the catheter 110 can be measured against calibrated force magnitude values. Exemplary electrical property changes measured in such embodiments may include the resonant frequency, electrical impedance or decay time constant. In other embodiments, changes in piezoelectric capacitance of the piezoelectric sensors 440, 442, 444 resulting from changes in the induced stress therein can be directly measured. The latter embodiments eliminate the requirement of providing an excitation signal from the control unit 120, but may have lower sensitivity relative to embodiments utilizing such excitation signals.

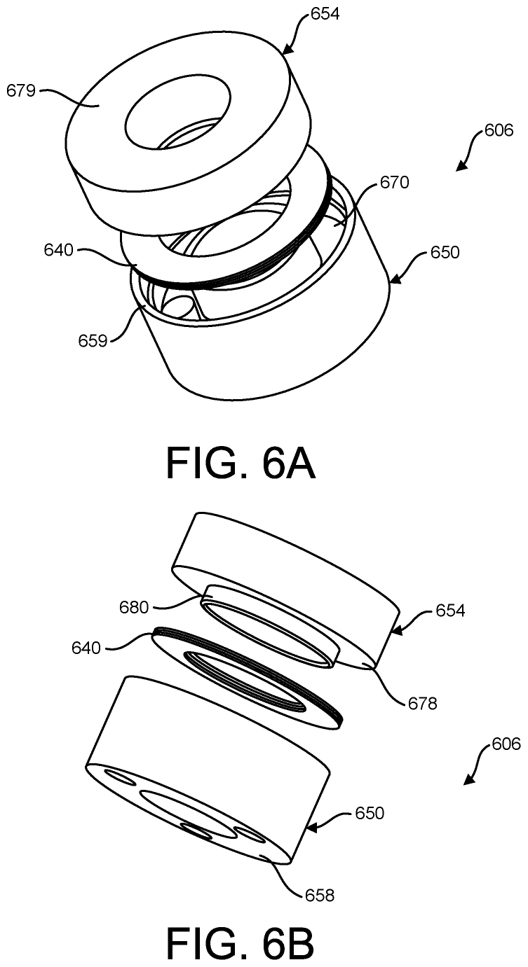

FIGS. 5A, 5B and 5C are perspective, elevation and cross-sectional views, respectively, of a force sensing mechanism 506 for use in the catheter 110, according to embodiments of the present disclosure. The force sensing mechanism 506 corresponds functionally to the force sensing mechanism 306 described above. In the illustrated embodiment, the force sensing mechanism 506 includes a plurality (in this case, three) annular piezoelectric sensors 540, 542, 544, a rigid proximal housing 550 and a rigid distal housing 554. As shown, the piezoelectric sensors 540, 542, 544 are disposed in and attached to the proximal housing 550.

In the various embodiments, the proximal housing 550 is securely attached to the proximal segment (111 in FIG. 1, 304 in FIG. 3), and the distal housing 554 is securely attached to the distal segment (113 in FIG. 1, 306 in FIG. 3) of the catheters 110, 300 described above. Additionally, the proximal and distal housings 550, 554 are separated, in the absence of an external force on the distal segment of the respective catheter, by a gap 556, which defines the maximum axial movement of the distal segment relative to the proximal segment under the action of an external force. Also, in the illustrated embodiment, a lumen 557 extends longitudinally through both the proximal housing 550 and the distal housing 554 to accommodate passage of catheter components, e.g., conductor wires, irrigation tubing, and the like to the distal segment of the catheter.

FIG. 5C is a cross-sectional elevation view bisecting the force sensing mechanism 606 through the piezoelectric sensor 540. The particular structural details illustrated in FIG. 5C are representative of the arrangement of the piezoelectric sensors 542, 544. As shown in FIG. 5C, the proximal housing has a proximal face 558, an opposite distal face 559, a longitudinal cavity 560, and a distal recess 570 formed in the distal face 559, the recess 570 defining a shoulder 575. As further shown, the distal housing 554 has a proximal face 578, an opposite distal face 579, and includes an axial projection 580 extending proximally from the proximal face 578.

As shown in FIG. 5C, the piezoelectric sensor 540 includes an upper layer 588, a lower layer 590, and a piezoelectric layer 592 disposed therebetween. The annular shape of the piezoelectric sensor 540 further defines a fixed portion 594 corresponding to the outer circumferential region of the piezoelectric sensor 540, and a free portion 596 that is radially inward of the fixed portion 594 and corresponds to the inner radial region of the piezoelectric sensor 540. As further shown, the annular shape of the piezoelectric sensor defines an aperture 597 generally centrally located through the piezoelectric sensor 540. As further shown, an optional backing element 598 is disposed within the longitudinal cavity 560 adjacent to the lower layer 590. Although for ease of illustration only the piezoelectric sensor 540 is shown in detail, it is emphasized that each of the piezoelectric sensors 542 and 544 have the same structure as the piezoelectric sensor 540. Additionally, the overall construction of the force sensing mechanism 506 with respect to the location of the piezoelectric sensors 542, 544 is identical to that shown in FIG. 5C, i.e., a cross-sectional view bisecting the piezoelectric sensors 542, 544 will appear identical to the structures depicted in FIG. 5C, including the presence of a recess, longitudinal cavity and projection and the corresponding relationship to the respective piezoelectric sensor.

The upper and lower layers 588, 590, or at least portions thereof, are electrically conductive and function as electrode layers during the operation of the piezoelectric sensor 540. Additionally, the piezoelectric layer 592 includes a piezoelectric material, for example, a piezoelectric ceramic such as lead zirconate titanate (PZT) or a piezoelectric polymer such as polyvinylidene fluoride (PVDF). As is recognized in the electro-mechanical arts, piezoelectric sensors generate measurable electrical characteristics when subjected to mechanical stress.

With particular reference to FIG. 5C, the distal recess 570 is axially aligned with the longitudinal cavity 560, and the piezoelectric sensor 540 is positioned within the distal recess 570 of the proximal housing 550, with the fixed portion 594 positioned on the shoulder 575, and the free portion 596 extending over the longitudinal cavity 560. As further shown, the backing element 598 abuts the lower layer 590 along the free portion 596 of the piezoelectric sensor 540. Additionally, the axial projection 580 of the distal housing 554 is positioned over and spans across the aperture 597 such that the axial projection bears upon the upper layer 588 in the free portion 596 of the piezoelectric sensor 540. The piezoelectric sensors 542, 544 each have the same positional relationship and with, respectively, a distal recess, a shoulder and a longitudinal cavity in the proximal housing 550, and an axial projection on the distal housing 554 as shown in FIG. 5C with respect to the piezoelectric sensor 540. Additionally, in embodiments, an optional backing element is disposed in the longitudinal cavity adjacent to each piezoelectric sensor 542, 544 in the same manner shown in FIG. 5C.

In embodiments, the fixed portion 594 of the piezoelectric sensor 540 is rigidly attached to the shoulder 575 of the proximal housing 550, and the free portion 596 of the piezoelectric sensor 540 extends over the longitudinal cavity 560. Because the rigid axial projection 580 of the distal housing 554 is in direct contact with the upper layer 588 of the piezoelectric sensor 540, forces applied to the distal segment 305 (FIG. 3) of the catheter will be transmitted to the free portion 596 of the piezoelectric sensor 540. Additionally, the arrangement of the free portion 596 extending over the longitudinal cavity 560 will maximize the piezoelectric effect exhibited by the piezoelectric sensor 540 in response to the transmitted force (i.e., the stress induced in the piezoelectric layer 592, and the sensor output resulting from the corresponding piezoelectric effect, will be substantially greater than in an arrangement where the piezoelectric sensor is rigidly fixed along its entire length).

In embodiments, the backing element 598, when present, is made of a compressible material (e.g., an elastomer) and functions to provide some support to the free portion 596 of the piezoelectric sensor 540 to resist deformation thereof when the external force is applied, while at the same time does not rigidly resist all such deformation. In embodiments, the mechanical properties of the backing element 598 can be tailored to fine tune the piezoelectric effect exhibited by the piezoelectric sensor 540.

As will be appreciated by the skilled artisan, because the piezoelectric sensor 540 is an electro-mechanical devices, electrical leads (not shown) are attached to the electrode structures on the upper and lower layers 588, 590. In embodiments, the electrical leads may be routed through the longitudinal cavity 560, may be routed to the lumen 557, or through some other access features within the proximal housing 550. As further shown in FIG. 5C, in embodiments, a potting material 599 may be disposed within open spaces within the proximal housing 550, including within the aperture 597 as shown, which can operate to hermetically seal the internal components of the catheter and the force sensing mechanism 506 and also to enhance the structural attachment between the proximal and distal segments of the catheter. In embodiments, the potting material 599 and the backing element 598 may be formed of the same compressible material, and may be formed in a single manufacturing step, although this is not strictly required. In some embodiments, the potting material 599 (or some other compressible material) may be disposed in the gap 556. In some embodiments, the backing element 598 and/or the potting material 599 may be omitted altogether.

It is emphasized again that the arrangement shown in FIG. 5C with respect to the piezoelectric sensor 540 is representative of the piezoelectric sensors 542 and 544.

Additionally, in embodiments, the piezoelectric sensors 540, 544 and 542 can have configurations other than the annular configurations shown. In particular, in embodiments, the piezoelectric sensors 540, 544 and 542 can be configured as substantially circular disks with no central aperture, and the respective free portions are defined by the central portions of the disks that are positioned over the corresponding longitudinal cavities.

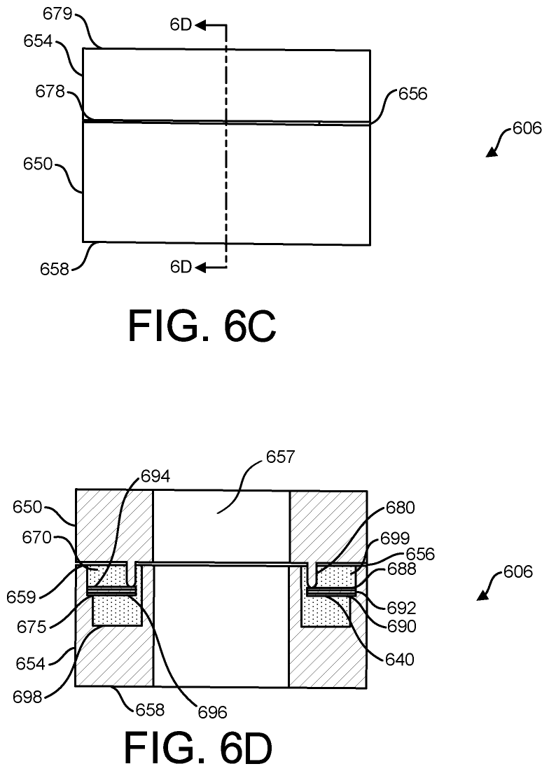

FIGS. 6A-6D illustrate an alternative force sensing mechanism 606 for use in the catheter 110, according to embodiments of the present disclosure. FIGS. 6A and 6B are perspective exploded views of the force sensing mechanism 606. FIG. 6C is an elevation view of the force sensing mechanism 606, and FIG. 6D is a cross-sectional elevation view of the force sensing mechanism 606 taken along the line 6D-6D in FIG. 6C.

The force sensing mechanism 606 corresponds functionally to the force sensing mechanism 306 described above. In the illustrated embodiment, the force sensing mechanism 606 includes a single annular piezoelectric sensor 640, a rigid proximal housing 650 and a rigid distal housing 654. As shown, the piezoelectric sensor 640 is disposed in and attached to the proximal housing 650.

In the various embodiments, the proximal housing 650 is securely attached to the proximal segment (111 in FIG. 1, 304 in FIG. 3), and the distal housing 654 is securely attached to the distal segment (113 in FIG. 1, 306 in FIG. 3) of the catheters 110, 300 described above. Additionally, the proximal and distal housings 650, 654 are separated, in the absence of an external force on the distal segment of the respective catheter, by a gap 656, which defines the maximum axial movement of the distal segment relative to the proximal segment under the action of an external force. Also, in the illustrated embodiment, a lumen 657 extends longitudinally through both the proximal housing 650 and the distal housing 654 to accommodate passage of catheter components, e.g., conductor wires, irrigation tubing, and the like to the distal segment of the catheter.

As shown, the proximal housing 650 has a proximal face 658, an opposite distal face 659, and a distal recess 670 formed in the distal face 659. As can be seen in the cross-sectional view of FIG. 6C, the distal recess 670 defines a shoulder 675. Additionally, the distal housing 654 has a proximal face 678, an opposite distal face 679, and an annular axial projection 680 extending proximally from the proximal face 678.

As shown in FIG. 6C, the piezoelectric sensor 640 includes an upper layer 688, a lower layer 690, and a piezoelectric layer 692 disposed therebetween. The annular shape of the piezoelectric sensor 640 further defines a fixed portion 694 corresponding to the outer circumferential region of the piezoelectric sensor 640, and a free portion 696 that is radially inward of the fixed portion 694 and corresponds to the inner radial region of the piezoelectric sensor 640. As further shown, an optional backing element 698 is disposed within the recess 670 adjacent to the lower layer 690.

The upper and lower layers 688, 690, or at least portions thereof, are electrically conductive and function as electrode layers during the operation of the piezoelectric sensor 640. Additionally, the piezoelectric layer 692 includes a piezoelectric material, for example, a piezoelectric ceramic such as lead zirconate titanate (PZT) or a piezoelectric polymer such as polyvinylidene fluoride (PVDF). As is recognized in the electro-mechanical arts, piezoelectric sensors generate measurable electrical characteristics when subjected to mechanical stress.

With particular reference to FIG. 6C, the piezoelectric sensor 640 is positioned with the fixed portion 694 positioned on the shoulder 675, and the free portion 696 extending over the lower portion of the recess 670. As further shown, the backing element 698 abuts the lower layer 690 along the free portion 696 of the piezoelectric sensor 640. Additionally, the axial projection 680 of the distal housing 654 is positioned such that the axial projection 680 bears upon the upper layer 688 in the free portion 696 of the piezoelectric sensor 640.

In embodiments, the fixed portion 694 of the piezoelectric sensor 640 is rigidly attached to the shoulder 675 of the proximal housing 650, and the free portion 696 of the piezoelectric sensor 640 extends over the recess 670. Because the rigid axial projection 680 of the distal housing 654 is in direct contact with the upper layer 688 of the piezoelectric sensor 640, forces applied to the distal segment 305 (FIG. 3) of the catheter will be transmitted to the free portion 696 of the piezoelectric sensor 640. Additionally, the arrangement of the free portion 696 extending over the longitudinal cavity 660 will maximize the piezoelectric effect exhibited by the piezoelectric sensor 640 in response to the transmitted force (i.e., the stress induced in the piezoelectric layer 692, and the sensor output resulting from the corresponding piezoelectric effect, will be substantially greater than in an arrangement where the piezoelectric sensor is rigidly fixed along its entire length).

In embodiments, the backing element 698, when present, is made of a compressible material (e.g., an elastomer) and functions to provide some support to the free portion 696 of the piezoelectric sensor 640 to resist deformation thereof when the external force is applied, while at the same time does not rigidly resist all such deformation. In embodiments, the mechanical properties of the backing element 698 can be tailored to fine tune the piezoelectric effect exhibited by the piezoelectric sensor 640.

As further shown in FIG. 6C, in embodiments, a potting material 699 may be disposed within open spaces within the proximal housing 650, including within the aperture 697 as shown, which can operate to hermetically seal the internal components of the catheter and the force sensing mechanism 606 and also to enhance the structural attachment between the proximal and distal segments of the catheter. In embodiments, the potting material 699 and the backing element 698 may be formed of the same compressible material, and may be formed in a single manufacturing step, although this is not strictly required. In some embodiments, the potting material 699 (or some other compressible material) may be disposed in the gap 656.

In operation, the annular piezoelectric sensor 640 is configured to operate similarly to each of the annular piezoelectric sensors 540, 542, 544 discussed elsewhere herein. However, because the force sensing mechanism 606 includes only a single piezoelectric sensor, the force sensing mechanism 606 is configured to sense only the magnitude (and not the direction) of an applied external force.

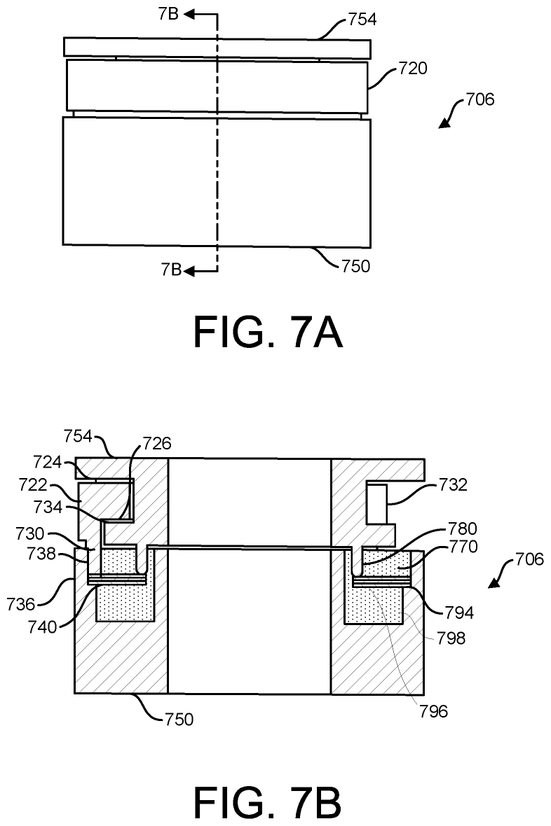

FIGS. 7A and 7B are elevation and sectional views, respectively, of an alternative force sensing mechanism 706 for use in the catheter of FIG. 3 in accordance with various embodiments of this disclosure. The force sensing mechanism 706 is in many respects constructed in the same manner as the force sensing mechanism 606 and includes a single, annular piezoelectric sensor 740, a proximal housing 750 and a distal housing 754 separated by a gap 756. The proximal housing 750 includes an annular recess 770, and the piezoelectric sensor 740 is disposed therein with a fixed portion 794 and a free portion 796 in the same manner as the piezoelectric sensor 640. Additionally, the distal housing 754 includes an annular axial projection 780 that bears upon the free portion 796 of the piezoelectric sensor 740 in the same manner as described above in connection with the other various embodiments. The illustrated embodiment further includes a backing element 798 supporting the free portion 796 of the piezoelectric sensor 740, although in other embodiments this backing element 798 can be omitted.

The force sensing mechanism 706 differs from the prior embodiments in that it further includes a semi-annular pre-load collar 720 operatively coupled to the proximal housing 750 and the distal housing 754. The pre-load collar 720 that is configured to facilitate applying an initial load to, and corresponding stress in, the piezoelectric sensor 740, or alternatively, to facilitate assembly of the force sensing mechanism 706 by ensuring intimate contact between the annular axial projection 780 and the piezoelectric sensor 740.

The pre-load collar 720 has a generally semi-circular shape that is complementary to the shape of proximal and distal housings 750, 754. As shown, the pre-load collar 720 has a distal body portion 722 having an upper surface 724 and a lower surface 726, and a shank portion 730 that extends proximally from the lower surface 726.

As further shown, in the embodiment of FIGS. 7A-7B, the distal housing 754 has an annular slot 732 extending radially inward about its circumference that is generally rectangular in shape to complement the cross-sectional shape of the distal body portion 722 of the pre-load collar 720. The annular slot 732 defines a radial bearing surface 734 adjacent to the lower surface 726 of the distal body portion 722 of the pre-load collar 720. Additionally, an annular wall 736 is defined in an outer perimeter of the proximal housing 750 radially outward of the recess 770.

In embodiments, the outer surface of the shank portion 730 is rotatably engaged with the inner surface of the annular wall 736 at a connection 738 (FIG. 7B). The connection 738, shown schematically in FIG. 7B is configured such that rotation of the pre-load collar 720 relative to the distal housing 754 causes the pre-load collar 720 to move axially relative to the proximal and distal housings 740, 754. In one exemplary embodiment, the connection 738 can be configured as a threaded connection comprising mating internal and external threads on the annular wall 736 and the shank portion 720, respectively.

FIG. 7B depicts the force sensing mechanism 706 in a substantially un-loaded state in which the projection 780 is in contact with but applying minimal or no force on the piezoelectric sensor 740, and the lower surface 726 of the body portion 722 of the pre-load collar 720 is axially spaced from the radial bearing surface 734 of the distal housing 754. As will be recognized by the skilled artisan, from the illustrated state, selective rotation of the pre-load collar 720 relative to the proximal housing 720, due to the configuration of the connection 738, causes the pre-load collar 720 to translate axially to move the lower surface 726 of the body portion 722 of the pre-load collar 720 into contact with the radial bearing surface 734 of the distal housing 734. Once the lower surface 726 of the body portion 722 of the pre-load collar 720 and the radial bearing surface 734 of the distal housing 734 are in contact, further rotation of the pre-load collar 720 in the same direction will urge the distal housing 754 toward the proximal housing 750 and cause the annular projection 780 to apply a force to the free portion 796 of the piezoelectric sensor 740. In this way, during assembly of the force sensing mechanism 706, the pre-load collar 720 can be operated to confirm the required contact between the projection 780 and the piezoelectric sensor 740, and if desired, apply a selective pre-load to facilitate tuning of the piezoelectric sensor 740.

The various embodiments of the present disclosure demonstrate significant advancements over conventional catheter force sensing technologies. In particular, the embodiments described herein eliminate the need for complex spring mechanisms utilized in the prior art catheters. Similarly, the piezoelectric sensors of the various embodiments are relatively low cost as compared to inductive position sensors utilized in the prior art catheters.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A catheter adapted to measure a contact force, the catheter comprising:
an elongate shaft having a proximal end and a distal end;
a distal end portion extending distally from the distal end of the shaft, the distal end portion defining a longitudinal axis extending therethrough and comprising:
a proximal segment;
a distal segment located distally of the proximal segment; and
a force sensing mechanism comprising:
a proximal housing fixed within the proximal segment;
a piezoelectric sensor mounted to the proximal housing and having a first portion fixedly secured to the proximal housing, and a second portion that is not fixedly secured to the proximal housing; and
a distal housing fixed within the distal segment and including a projection that contacts the second portion of the piezoelectric sensor and is configured to apply an axial force to the second portion of the piezoelectric sensor upon application of an external force to the distal segment.

2. The catheter of claim 1, wherein the proximal housing includes an upper surface, a lower surface, and a cavity extending from the lower surface through the upper surface, and wherein the first portion of piezoelectric sensor is fixedly attached to the upper surface, and wherein the second portion of the piezoelectric sensor extends at least partially across the cavity.

3. The catheter of claim 2, wherein the proximal housing comprises a compressible backing material disposed within a portion of the cavity and contacting the second portion of the piezoelectric sensor opposite the projection on the distal housing, wherein the backing material resists deformation of the second portion of the piezoelectric sensor when the external force is applied to the distal segment.

4. The catheter of claim 2, wherein the piezoelectric sensor has an arcuate or rectangular profile when viewed from a direction parallel to the longitudinal axis.

5. The catheter of claim 2, wherein piezoelectric sensor has an annular shape when viewed from a direction parallel to the longitudinal axis, and wherein the first portion is an outer circumferential portion of the piezoelectric sensor, and the second portion is located radially inward of the first portion of the piezoelectric sensor.

6. The catheter of claim 2, wherein the piezoelectric sensor is a generally circular disk, and wherein the second portion of each piezoelectric sensor extends across the respective cavity.

7. The catheter of claim 1, further comprising a pre-load mechanism operatively coupled to the proximal housing and configured to allow a user to selectively pre-load the piezoelectric sensor.

8. A catheter adapted to measure a contact force, the catheter comprising:
an elongate shaft having a proximal end and a distal end;
a distal end portion extending distally from the distal end of the shaft, the distal end portion defining a longitudinal axis extending therethrough and comprising:
a proximal segment;
a distal segment located distally of the proximal segment; and
a force sensing mechanism comprising:
a proximal housing fixed within the proximal segment, the proximal housing having a lower surface and an upper surface;
a plurality of piezoelectric sensors mounted to and circumferentially spaced from one another about the proximal housing, each piezoelectric sensor having a first portion fixedly secured to the proximal housing, and a second portion that is not fixedly secured to the proximal housing; and
a distal housing fixed within the distal segment and including a plurality of projections, each of the projections contacting the second portion of a respective one the piezoelectric sensors and being is configured to apply an axial force to the second portion of the respective piezoelectric sensor upon application of an external force to the distal segment,
wherein each of the plurality of piezoelectric sensors is configured to generate an output indicative of an amount of the axial force applied to the second portion thereof in response to the external force applied to the distal segment.

9. The catheter of claim 8, wherein the proximal housing comprises a plurality of cavities extending from the lower surface through the upper surface, each of the cavities being aligned with a respective one of the piezoelectric sensors, wherein the second portion of each of the piezoelectric sensors extends at least partially across a respective one of the cavities.

10. The catheter of claim 9, wherein the proximal housing comprises a compressible backing material disposed within each of the cavities and contacting the second portion of the piezoelectric sensor positioned thereover opposite the respective projection on the distal housing, wherein the backing material resists deformation of the second portion of the piezoelectric sensor when the external force is applied to the distal segment.

11. The catheter of claim 9, wherein each piezoelectric sensor has an annular shape when viewed from a direction parallel to the longitudinal axis, and wherein the first portion is an outer radial portion of the piezoelectric sensor, and the second portion is an inner radial portion of the piezoelectric sensor.

12. The catheter of claim 9, wherein each piezoelectric sensor is a generally circular disk, and wherein the second portion of each piezoelectric sensor extends across the respective cavity.

13. The catheter of claim 9, wherein the force sensing mechanism comprises three piezoelectric sensors mounted to the proximal housing and circumferentially spaced about the longitudinal axis, each of the piezoelectric sensors having a first portion fixedly secured to the proximal housing, and a second portion that is deflectable relative to the first portion, and wherein the distal housing comprises three projections, each projection contacting the second portion of a respective one of the piezoelectric sensors and configured to apply an axial force to the second portion of the respective piezoelectric sensor upon application of the external force to the distal segment.

14. The catheter of claim 13, wherein the proximal housing comprises three cavities extending proximally from the upper surface, each of the cavities being aligned with a respective one of the piezoelectric sensors, wherein the second portion of each of the piezoelectric sensors extends at least partially across a respective one of the cavities.

15. A force sensing mechanism for an ablation catheter, the force sensing mechanism adapted to measure a contact force and comprising:
a proximal housing having a lower surface and an upper surface;
a plurality of piezoelectric sensors mounted to and circumferentially spaced from one another about the proximal housing, each piezoelectric sensor having a first portion fixedly secured to the proximal housing, and a second portion that is not fixedly secured to the proximal housing; and
a distal housing including a plurality of projections, each of the projections contacting the second portion of a respective one the piezoelectric sensors and being configured to apply an axial force to the second portion of the respective piezoelectric sensor upon application of an external force to the distal housing,
wherein each of the plurality of piezoelectric sensors is configured to generate an output indicative of an amount of the axial force applied to the second portion thereof in response to the external force applied to the distal housing.

16. The force sensing mechanism of claim 15, wherein the proximal housing comprises a plurality of cavities extending proximally from the upper surface, each of the cavities being aligned with a respective one of the piezoelectric sensors, wherein the second portion of each of the piezoelectric sensors extends at least partially across a respective one of the cavities.

17. The force sensing mechanism of claim 16, wherein the proximal housing comprises a compressible backing material disposed within each of the cavities and contacting the second portion of the piezoelectric sensor positioned thereover opposite the respective projection on the distal housing, wherein the backing material resists deformation of the second portion of the piezoelectric sensor when the external force is applied to the distal segment.

18. The force sensing mechanism of claim 15, wherein each piezoelectric sensor has a rectangular shape.

19. The force sensing mechanism of claim 15, wherein each piezoelectric sensor has an annular shape, wherein the first portion is an outer circumferential portion of the piezoelectric sensor, and the second portion is located radially inward of the first portion of the piezoelectric sensor.

20. The force sensing mechanism of claim 15, wherein each piezoelectric sensor is a generally circular disk, and wherein the second portion of each piezoelectric sensor extends across the respective cavity.
